(19) United States
(12) Patent Application Publication
(10) Pub. No.: US 2024/0252035 A1
(43) Pub. Date: Aug. 1, 2024

(54) METHOD FOR FITTING VIRTUAL GLASSES

(71) Applicant: ACEP FRANCE, PARIS (FR)

(72) Inventors: Benoit GRILLON, ORLEANS (FR); Jean-philippe SAYAG, PARIS (FR)

(73) Assignee: ACEP FRANCE, PARIS (FR)

(21) Appl. No.: 18/564,709

(22) PCT Filed: Jun. 17, 2022
(86) PCT No.: PCT/EP2022/066607

§ 371 Date: Nov. 28, 2023

(30) Foreign Application Priority Data
Jun. 18, 2021 (FR) FR2106482

(51) Int. Cl. A61B 3/028; A61B 3/00; A61B 3/14; G01S 7/4865; G02C 13/00

(52) U.S. Cl. CPC A61B 3/028; A61B 3/0041; A61B 3/14; G01S 7/4865; G02C 13/005

(57) ABSTRACT

A method for fitting virtual glasses including the steps of: —[100] placing a real pair of glasses on a user's face; —[200] measuring the position of said real pair of glasses relative to said user's face by a measuring device; —[300] after removing said real pair of glasses, mapping the user's face using a 3D mapping element; —[400] constructing a 3D model of the user's face and of said virtual pair of glasses; —[500] measuring the ophthalmological parameters of said user from the 3D model of the user's face constructed in step [400].

### FIELD

The present invention relates to a method for fitting a virtual pair of glasses and to a method for measuring the parameters for the preparation of an ophthalmic lens for mounting thereof in a real pair of glasses similar to the virtual pair of glasses being tested.

### BACKGROUND

After the prescription of corrective lenses by the ophthalmologist, the patient selects a frame in which the lenses will be mounted.

Usually, the selection of frames is done in an optician who will then measure different parameters specific to the frame, the face of the patient and its attitude in near-sight and/or far-sight. Among these parameters, mention may be made in particular of the lens-eye distance, the camber angle of the frame, the angle of inclination of the frame, the pupillary spacings and the pupillary heights. The measurement of these parameters allows preparing ophthalmic lenses perfectly adapted to the frame and to the patients.

Recently, devices have been developed to automatically calculate the ophthalmic parameters based on images of the face of the user wearing the frame. These devices are in the form of fixed columns or portable tablets comprising at least one camera and a computer capable of processing the image captured by the camera in order to calculate the desired parameters. Often, the tested frame is temporarily associated with a reference frame with a predefined size allowing improving the accuracy of the measurement.

Since several years, the actual fitting of the frame in the optician has been competing with means for fitting virtual frames in a shop or at home. Typically, these fitting means comprise a camera which captures in real-time the image of the user and transmits it, associated with a virtual frame, on a display screen. These means are associated with a computer program which is capable of extracting data such as the position of the face from the image of the user, the position of the eyes of the user and computing a virtual scene comprising the face of the user on which a pair of 3D glasses selected by the user in a database is superimposed.

This virtual fitting can be completely delocalised because it is possible to implement it on the smartphone or the computer of the user. The number of available frames is no longer limited by the size of the displays of the optician but by the capacity of the servers. Hence, the client could select from thousands of frames at any time of day or night.

Nonetheless, once the virtual frame has been selected, it is still necessary to measure the ophthalmic parameters of the user, which involves returning the client to the optician where he/she will have to wear a real pair of glasses equivalent to the virtual pair of glasses.

Devices allowing avoiding this passage in the optician have already been proposed. For example, it has been considered to directly calculate the ophthalmic parameters based on the 3D scene created during the virtual test. However, the position of the virtual glasses in the 3D scene is coarse and does not allow performing accurate calculations.

For the same frame, each wearer will place his/her glasses differently depending on the position of his/her ears, the shape of his/her nose and the heights of his/her cheekbones. Hence, it is impossible to use this scene as such for the calculation of ophthalmic parameters.

The present invention aims to solve this problem by providing a method which enables the accurate calculation of the ophthalmic parameters of a frame wearer simultaneously with the virtual fitting of these frames. The method according to the invention also allows improving the accuracy of the position of the virtual frame during the test.

### SUMMARY

The present invention relates to a method for fitting virtual glasses comprising the steps of:
[100] placing on the face of a user a real pair of glasses,
[200] measuring the position of said real pair of glasses relative to the face of said user by means of a measuring device,
[300] mapping the face of the user by a 3D mapping means,
[400] constructing a 3D model of the face of the user and of said virtual pair of glasses taking into account the position measured in step [200], and
[500] measuring the ophthalmological parameters of said user based on the 3D model of the face of the user constructed in step [400]. The methods of the prior art consist in creating a 3D model in which the virtual glasses are arbitrarily placed at the surface of the face of the user. In the prior art, the lower portion of the bridge of the frame is systematically placed on the highest portion of the nasal edge with the rear face of said bridge in contact with the forehead of the user. This position does not necessarily correspond to the position usually used by the user and does not allow accurately calculating the ophthalmic parameters.

On the contrary, in the method according to the invention, the 3D model of the face of the user and of said virtual pair of glasses is created while taking into account the way in which the user wears a real pair of glasses in normal condition. Consequently, irrespective of the virtual pair of glasses, the latter will be placed in the 3D model exactly as the user would wear the equivalent real pair of glasses. Thus, it is possible to accurately calculate the ophthalmic parameters directly from the 3D model.

By “while taking into account the position measured in step [200]”, it should be understood that in the 3D model constructed in step [400] the position of the virtual pair of glasses, with respect to the representation of the face of the user, reproduces all or part of that measured in step [200]. Preferably, in the 3D model constructed in step [400] the position of the virtual pair of glasses, with respect to the representation of the face of the user, reproduces at any point that measured in step [200].

According to a preferred embodiment, the mapping of the face of the user of step [300] is carried out after having pulled off said real pair of glasses from the face of the user.

According to a preferred embodiment, the method according to the invention further comprises the steps of:
[310] capturing the image of the face of the user by an image capture means,
[320] identifying, on the image captured in step [310], the position of the pupils of said user,
[340] integrating the position of the pupils, determined in step [320] to the 3D model established in step [400].

In the context of the present invention, the position of the pupils may be replaced by the position of the corneal reflections. Thus, the terms “position of the pupils” and “position of the corneal reflections” may be used interchangeably.

According to a preferred embodiment, the method according to the invention further comprises step [210] of saving said position of said real pair of glasses relative to the face of said user, associated with a specific identifier of said user.

According to a preferred embodiment, the method according to the invention comprises step [600] of displaying, on a display means, a virtual pair of glasses on the image of the face of said user captured in step [310], characterised in that said virtual pair of glasses is positioned so as to reproduce the position measured in step [200].

In the context of the latter embodiment, step [200] may be carried out by the user or by another person who will compare the location of the virtual pair of glasses displayed in step [600] at the position of the real pair of glasses that the user wears, and to provide information for making said display means coincide with said real pair of glasses and said virtual pair of glasses.

According to a preferred embodiment, the ophthalmological parameters measured in step [340] are selected from the group consisting of the pupillary heights, the pantoscopic angle, the horizontality of the frame, the horizontality of the lenses with respect to said frame, the pupillary deviations in far-sight and/or near-sight, the camber of said frame and the position of the centring marks on each of the lenses.

According to a preferred embodiment, the measurement of the position of said real pair of glasses relative to the face of said user comprises determining the point of contact between said real pair of glasses and the edge of the nose of said user.

According to a preferred embodiment, the ophthalmological parameters measured in step [340] are selected from the group consisting of the pupillary heights, the pantoscopic angle, the horizontality of the frame, the horizontality of the lenses with respect to said frame, the pupillary deviations in far-sight and/or near-sight, the camber of said frame, the lens-eye distance and the position of the centring marks on each of the lenses.

According to a preferred embodiment, the measurement of the position of said real pair of glasses relative to the face of said user comprises determining the point(s) of contact between said real pair of glasses and the edge of the nose said user.

According to a still more preferred embodiment, the measurement of the position of said real pair of glasses on the face of said user further comprises determining the point(s) of contact between said real pair of glasses and each of the ears of said user.

According to a preferred embodiment, said measuring device used in step [200] comprises a means capable of measuring the distance.

According to a preferred embodiment, said means capable of measuring the distance is an ultrasound sensor, a time-of-flight camera, a truedepth camera, a LIDAR.

According to a preferred embodiment, said means capable of measuring the distance is associated with a computing means capable of constructing a depth map of the face of the user and of the real pair of glasses.

According to a preferred embodiment, said image capture means used in step [310] is a video sensor.

According to a preferred embodiment, said display means used in step [600] is a video screen.

According to a preferred embodiment, said image capture means and said display means are included in a tablet or a smartphone.

According to a preferred embodiment, said measuring device, said image capture means and said display means are included in a tablet.

The present invention also relates to a device comprising an image capture sensor, a display means, a measuring device and a computing means implementing a computer program implementing a method according to the invention.

The steps of the method according to the invention may be implemented in an order different from that set out hereinabove. For example, step [300] may be carried out prior to step [100] or step [200].

### DETAILED DESCRIPTION

In the context of the present invention, step [100] consists in placing a real pair of glasses on the face of a user.

By “real pair of glasses”, it should be understood any reinforcement composed of one face and of two temples. Said face further comprises a bridge intended to rest on the nose of the user. The presence of lenses at said face is not mandatory. The term “real” is used in opposition to the term “virtual”. Thus, said real pair of glasses is made of physical materials and can be touched.

Said real pair of glasses is not necessarily a pair of glasses intended to receive corrective lenses. For example, it may consist of a structure constructed for use only in the context of the method according to the invention.

According to one embodiment, said real pair of glasses is selected from the group consisting of all frames available on the market.

According to another preferred embodiment of the invention, said real pair of glasses is a glasses frame at the surface of which are arranged, in a temporary or definitive manner, markings with predefined sizes and positions. The presence of these markers allows facilitating the detection of the frame and its position. For example, these marks may consist of geometric shapes with a colour different from those of the rest of the frame.

In the context of the present invention, by “position of said real pair of glasses relative to the face of said user”, it should be understood at least the location, on the nose of the user, of the point(s) of contact between the bridge of said real pair of glasses and the nose of the user. Preferably, said position further comprises the location, on the ears of the user, of the point(s) of contact between the temples of said real pair of glasses and the nose of the user. Still more preferably, said position further comprises the location, on the temples of the real pair of glasses, of the contact point(s) between the temples of said real pair of glasses and the ears of the user.

Said position is defined in space with respect to a reference frame. The nature of said reference frame is not relevant in the context of the present invention. For example, a reference frame (O, \vec{i}, \vec{j}, \vec{k}) may be defined using four fixed points of the face of the user. Advantageously, these fixed points are selected from the group consisting of the inner corner of the left eye, the inner corner of the right eye, the outer corner of the left eye, the outer corner of the right eye, the tip of the nose and the tip of the chin of the user.

Said “measuring device” may be selected from among the numerous devices accessible to a person skilled in the art. For example, said measuring device may be a set of manual measuring means such as rulers, compass and/or calliper.

In the case where the method according to the invention comprises step [600] of displaying, on a display means, a virtual pair of glasses on the image of the face of said user captured in step [310], said measuring device may comprise an interface on which the user or another person will be able to enter information intended to make said real pair of glasses and said virtual pair of glasses coincide on said display means.

In particular, said interface may be a keyboard, a joystick, a mouse or a touchscreen. According to a preferred embodiment of the invention, said interface is a touchscreen which serves as a display means in step [600].

In the context of this embodiment, a first 3D model of the face of the user and of said virtual pair of glasses, positioned arbitrarily, is made from the 3D mapping obtained in step [300]. By “arbitrarily”, it should be understood that said virtual pair of glasses could be placed indifferently at any location of the first 3D model. Nonetheless, advantageously, said virtual pair of glasses is placed so that at least the bridge of said virtual pair of glasses is in contact with the edge of the nose of the face of the user.

Afterwards, said virtual pair of glasses is displayed, on said display means, on the image of the face of said user captured in step [310].

Thus, the user or another person will be able to compare the location of the displayed virtual pair of glasses with that of the real pair of glasses that the user wears, and enter correction information (for example, upwards, downwards, front-rear rotation, left-right rotation) via said interface. Said correction information is used to correct the position of the virtual pair of glasses in said 3D model. When the display of the real pair of glasses and that of the virtual pair of glasses perfectly coincides, it could be assumed that the position of the virtual pair of glasses in the 3D model corresponds to the position of said real pair of glasses relative to the face of said user as sought in step [200].

Advantageously, said measuring device used in step [200] is a means capable of creating a 3D mapping of the face of the user and of the worn real pair of glasses. The 3D mapping may be obtained by a set of several equipment components working together to scan the environment continuously and process the obtained signal to establish said 3D mapping.

In the context of step [200], by “face of the user”, it should be understood at least one portion of the front face of the skull of the user comprising at least the eyes, the edge of the nose and the junction point between the upper portion of the ear and the rest of the skull. Advantageously, the face of the user mapped in step [200] comprises at least the area of the face delimited by the top of the forehead, the chin and the rear of the ears of the user.

Said means capable of creating a 3D mapping of the face of the user may consist of any type of 3D scanner suitable for digitising a portion of the body of an individual. For example, the 3D scanner may be a hand-held 3D scanner. In some embodiments, the 3D scanner uses at least two 2D images to create the 3D scan. For example, the 3D scanner may comprise a plurality of cameras which capture several 2D images of the face of the user. Afterwards, the 3D scanner superimposes and assembles the 2D images. Each point on the 2D images is mathematically triangulated to reconstruct the scale and the location in space in order to create the 3D scan. The larger the number of superimposed 2D images, the higher the resolution and the reliability of the resulting 3D scan will be.

In other embodiments, the 3D scanner may comprise one single camera as well as additional equipment which enables it to correctly capture multiple images of the face of the user which can be used to create 3D image of the face of the user. For example, the 3D scanner may comprise a camera as well as mirrors arranged such that the camera could capture several images of the face of the user. In another example, the 3D scanner may comprise one single camera as well as a mechanism allowing moving the camera towards another location so that the camera could capture several images of the face of the user from different locations. In still another example, the 3D scanner may comprise one single camera as well as an interactive interface which indicates to the user, by means of indicators or other mechanisms, how to move the camera to capture several images.

In another embodiment, the 3D scanner may comprise one single camera as well as a structured light projector. The structured light projector may project a grid or pattern over the face of the user which could be collected by the camera and decoded to provide absolute dimensional distances of the captured object in order to create a 3D scan of the face of the user.

In this latter embodiment, said means capable of creating a 3D mapping used in step [200] may comprise a first projection means capable of projecting an optical radiation pattern over the face of the user. According to a preferred embodiment, the optical radiation used to this end is typically in the infrared (IR) range.

In order to capture an image of the patient with the optical radiation pattern, said means capable of creating a 3D mapping used in step [200] further comprises a first camera. Preferably, the optical axis of the first projection means is parallel to the optical axis of said first camera. In addition, said first camera comprises a sensor capable of recording the optical radiation emitted by said first projection means.

In order to process the image recorded by said first camera, said means capable of creating a 3D mapping used in step [200] further comprises a processing device capable of processing the image of the pattern in order to generate a 3D mapping.

According to a preferred embodiment of the invention, said measuring device comprises a means capable of measuring the distance selected from the group consisting of ultrasonic sensors, time-of-flight cameras, truedepth cameras and LIDAR.

According to an even more preferred embodiment of the invention, said means capable of measuring the distance is associated with a computing means capable of constructing a depth map of the face of the user and of the real pair of glasses.

In addition, the means capable of creating a 3D mapping is advantageously associated with a processing device capable of extracting from said 3D mapping the location, on the nose of the user, of the point(s) of contact between the bridge of said real pair of glasses and the nose of the user and advantageously the location, on the ears of the user, of the point(s) of contact between the temples of said real pair of glasses and the nose of the user and even more advantageously the location, on the temples of the real pair of glasses, of the point(s) of contact between the temples of said real pair of glasses and the nose of the user.

According to a preferred embodiment, the processing device is capable of identifying portions of the face of the patient such as the inner corner of the eyes, the centre of the pupils, the middle of the forehead, and/or the middle of the chin and determining their 3D location.

This set of equipment capable of producing a 3D mapping is provided, for example, in a smartphone or a tablet. In this respect, the method according to the invention is implemented by a smartphone or a tablet. In this embodiment, the method according to the invention is more particularly implemented by software dedicated to a smartphone or a tablet.

According to a preferred embodiment, the method according to the invention further comprises step [210] of saving said position associated with a specific identifier of said user, in a database.

By “specific identifier”, it should be understood any single or plural means allowing identifying a user. Among these, mention may be made in particular of the civil status, the social security number, the passport number, the address, the birth date, the client number, the anthropometric data and combinations thereof. According to a preferred embodiment, said specific identifier is an anthropometric piece of data of the user and even more preferably an anthropometric piece of data obtained based on the 3D mapping of its face. Any other specific identifier may be created for use in the context of the method according to the invention.

By “backup”, it should be understood the preservation of said information on any storage medium. For example, the suitable storage media are the physical electronic and electromagnetic information carriers (for example, hard disk) or virtual (for example, the cloud). According to a preferred embodiment of the invention, said storage medium is accessible remotely via the Internet network.

Said specific identifier is associated with said position such that reading of the storage medium allows finding out the position of said real pair of glasses relative to the face of a specific user.

Step [300] of mapping the face of the user, after having pulled off said real pair of glasses, by a 3D mapping means, may be carried out by a means identical to or different from said measuring device used in step [200].

According to a preferred embodiment, the method according to the invention further comprises step [310] of capturing the image of the face of the user, after having pulled off said real pair of glasses, by an image capture means.

Preferably, by “image capture means”, it should be understood a photographic sensor (for example, CCD, CMOS). Said sensor is associated with means for processing the signal of said sensor capable of creating a digital image. In the case where the measuring device used in step [200] comprises an image capture means, the latter may be identical to or different from said image capture means used in step [310].

The capture of the image of the face of the user is done punctually or continuously.

Said image capture means is provided, for example, in a smartphone or a tablet. In this respect, the method according to the invention is implemented by a smartphone or a tablet. In this embodiment, the method according to the invention is implemented more particularly by software dedicated to a smartphone or a tablet.

According to a preferred embodiment, the method according to the invention further comprises step [320] of identifying, on the image of the face of the user captured in step [310], the position of the pupils of said user. Different algorithms may be implemented for carrying out this step. These algorithms may implement successive steps of detecting the face of the user, then detecting the eyes of the user in the previously identified area and finally detecting the pupils of the user. Among these algorithms, mention may be made of those described by Ciesla et al. (Ciesla, M., & Koziol, P. (2012). Eye Pupil Rental Using Webcam. ArXiv, abs/1202.6517).

In step [340], the position of the pupils, determined in step [320], is integrated into the 3D model established in step [400].

By “integrated”, it should be understood that said 2D position of the pupils measured based on the image captured in step [310] is transformed into a 3D position in the reference frame of the 3D mapping established in step [300] then integrated into the 3D model. This operation may be performed simply by taking into account the 3D positions of the corners of the eyes and of the apex of the cornea.

According to a preferred embodiment, the method according to the invention further comprises step [400] of constructing a 3D model of the face of the user and of said virtual pair of glasses.

The construction of a 3D model of the face of the user and of said virtual pair of glasses performed in step [400] may be carried out by any 3D modelling system known to a person skilled in the art. This construction will lead to obtaining a 3D scene (cf. for example https://fr.wikipedia.org/wiki/Scene_3D) comprising at least the face of the face of the user and the selected virtual pair of glasses.

The software allowing the construction of a 3D model are well-known to a person skilled in the art, among these, mention may be made in particular of Ogre3D, Autodesk 3ds Max, Autodesk Maya, MotionBuilder, Autodesk Softimage, Bryce 3D, Carrara, Cinema 4D, Dynamation, Houdini, LightWave 3D, MASSIVE, Messiah, Moviestorm, Softimage 3D, Strata 3D, Swift 3D.

In the context of the present invention, said construction of a 3D model may comprise no implementation of a rendering engine. Indeed, the primary purpose of this modelling is the calculation of the relative positions of the elements of said virtual pair of glasses with respect to the face of the user. Hence, it is not necessary to calculate the rendering of this scene and display it (except in the case where the method according to the invention further comprises step [600]).

In the context of the present invention, the term “said virtual pair of glasses” refers to a pair of glasses which is displayed in the virtual environment but does not form part of the physical world.

Many virtual pairs of glasses are commercially available. There are many databases containing several thousand virtual glasses in which the user could select prior to step [400].

In practice, a virtual pair of glasses is defined by a collection of points in the 3D space, connected by various geometric entities such as triangles, lines, curved surfaces, etc. This collection of points may be supplemented by information relating to the colour, to the texture and to the reflectance of the different surfaces of said virtual pair of glasses. Being a collection of data (points and other information), the virtual pairs of glasses may be created manually, algorithmically or by scanning. This set of information may be combined in one or more computer file(s). Advantageously, said files are stored on one or more storage media. Preferably, said storage medium is accessible remotely via the Internet network.

In the case where the method according to the invention does not comprise step [600], said virtual pair of glasses may comprise no textures, since only the 3D geometric shape of said virtual pair of glasses is necessary for the calculation of the ophthalmic parameters.

Step [500] consists in measuring the ophthalmological parameters of the user based on the 3D model of the face of the user constructed in step [400]. Among the ophthalmological parameters measured in step [500], mention may be made of the pupillary heights, the pantoscopic angle, the horizontality of the frame, the horizontality of the lenses with respect to said frame, the pupillary deviations in far-sight and/or near-sight, the lens-eye distance, the camber of said frame, the head-eye coefficient, the lens-eye distance and the position of the centring marks on each of the lenses.

For example, based on the 3D model calculated in step [400], it is easy to calculate the pupillary heights based on the position of the pupil of the user with respect to that of the cerclage of the frame surrounding the lens.

In the same manner, the lens-eye distance may be calculated based on the position, in the 3D model, of the apex of the cornea and of the lenses (or of their position deduced from the position of the cerclage).

The pantoscopic angle may be calculated by measuring the angle of the face of the virtual pair of glasses with respect to the vertical plane.

All these calculations may be done by an operator who can designate, via a computer interface, the different points (apex of the cornea, position of the lenses, etc.) necessary for the calculation of the ophthalmic parameters. Advantageously, the calculation may be automated based on the points identified by the operator.

Alternatively, the entirety of step [500] is carried out automatically by an information processing means programmed so as to identify within said 3D model the points necessary for the calculation of the ophthalmic parameters and for the calculation thereof.

Step [600] of displaying, on a display means, a virtual pair of glasses on the image of the face of said user entered in step [310], characterised in that said virtual pair of glasses is positioned so as to reproduce the position measured in step [200].

In the context of the present invention, by “display means”, it should be understood any means enabling the display of images and more particularly of digital images.

The creation of a digital image comprising a virtual pair of glasses on the image of the face of said user based on said 3D model created in step [400] is a 3D computer graphics conventional step. Preferably, step [500] implements a 3D rendering engine. A 3D rendering engine may be conventional software or algorithms integrated in special graphics cards which compute one or more 3D image(s) by rendering therein the 3D projection, the textures and the lighting effects, inter alia.

The 3D rendering engine analyses the elements of a digitised image (colours, intensity and type of light, shadows and combinations thereof, etc.), which image is supposed to be seen by a virtual “camera”, the x y z coordinates of which determine the view angle and the position of the objects.

Among the 3D rendering engines that can be used in the context of the present invention, mention may be made in particular of Arnold, Aqsis, Arion Render, Artlantis, Atomontagel, Blender, Brazil r/s, BusyRay, Cycles, Enscape, FinalRender, Fryrender, Guerilla Render2, Indigo, Iray, Kerkythea, KeyShot, Kray, Lightscape, LightWorks, Lumiscaphe, LuxRender, Maxwell Render, Mental Ray, Mitsuba3, Nova, Octane4, POV-Ray, RenderMan, Redsdk, Redway3d, Sunflow, Turtle, V-Ray, VIRTUALIGHT and YafaRay.

Numerous methods allowing implementing step [600] of displaying, on a display means, a virtual pair of glasses on the image of the face of said user captured in step [310] are described in the prior art. Among these, mention may in particular be made of the methods described in the documents WO013207 and EP2526510 or commercial software such as SmartMirror®.

The entire method according to the invention may be implemented by software implemented in a smartphone, a tablet or a computer.

Thus, the present invention also relates to a smartphone, a tablet or a computer remarkable in that it implements a method according to the invention.

1-14. (canceled)

15. A method for fitting virtual glasses comprising the steps of:
[100] placing on the face of a user a real pair of glasses,
[200] measuring the position of said real pair of glasses relative to the face of said user by means of a measuring device,
[300] mapping the face of the user by a 3D mapping means,
[400] constructing a 3D model of the face of the user and of said virtual pair of glasses taking into account the position measured in step [200], and
[500] measuring the ophthalmological parameters of said user based on the 3D model of the face of the user constructed in step [400].

16. The method for fitting virtual glasses according to claim 15, further comprising the steps of:
[310] capturing the image of the face of the user by an image capture means,
[320] identifying, on the image captured in step [310], the position of the pupils of said user,
[340] integrating the position of the pupils, determined in step [320] to the 3D model established in step [400].

17. The method for fitting virtual glasses according to claim 15, further comprising step [210] of saving said position associated with a specific identifier of said user.

18. The method for fitting virtual glasses according to claim 16, further comprising step [600] of displaying, on a display means, a virtual pair of glasses on the image of the face of said user captured in step [310], wherein said virtual pair of glasses is positioned so as to reproduce the position measured in step [200].

19. The method for fitting virtual glasses according to claim 15, wherein the ophthalmological parameters measured in step [500] are selected from the group consisting of the pupillary heights, the pantoscopic angle, the horizontality of the frame, the horizontality of the lenses with respect to said frame, the pupillary deviations in far-sight and/or near-sight, the camber of said frame, the lens-eye distance and the position of the centring marks on each of the lenses.

20. The method for fitting virtual glasses according to claim 15, wherein the measurement of the position of said real pair of glasses relative to the face of said user comprises determining the point of contact between said real pair of glasses and the edge of the nose of said user.

21. The method for fitting virtual glasses according to claim 15, wherein the measurement of the position of said real pair of glasses on the face of said user comprises determining the point of contact between said real pair of glasses and each of the ears of said user.

22. The method for fitting virtual glasses according to claim 15, wherein said measuring device comprises a means capable of measuring the distance.

23. The method for fitting virtual glasses according to claim 22, wherein said means capable of measuring the distance is an ultrasound sensor, a time-of-flight camera, a truedepth camera, a LIDAR.

24. The method for fitting virtual glasses according to claim 22, wherein said means capable of measuring the distance is associated with a computing means capable of constructing a depth map of the face of the user and of the real pair of glasses.

25. The method for fitting virtual glasses according to claim 18, wherein said image capture means is a video sensor.

26. The method for fitting virtual glasses according to claim 25, wherein said display means is a video screen.

27. The method for fitting virtual glasses according to claim 18, wherein said image capture means and said display means are included in a tablet.

28. The method for fitting virtual glasses according to claim 18, wherein said measuring device, said image capture means and said display means are included in a tablet.
